(19) United States
(12) Patent Application Publication
(10) Pub. No.: US 2026/0363805 A1
(43) Pub. Date: Nov. 19, 2026

(54) System and computer-implemented method for controlling at least one functionality of a surgical instrument

(71) Applicant: KARL STORZ SE & Co. KG, Tuttlingen (DE)

(72) Inventor: Lena Felber, Emmingen-Liptingen (DE)

(73) Assignee: KARL STORZ SE & Co. KG, Tuttlingen (DE)

(21) Appl. No.: 19/141,187

(22) PCT Filed: Dec. 18, 2023
(86) PCT No.: PCT/EP2023/086341

§ 371 Date: Jun. 19, 2025

(30) Foreign Application Priority Data
Dec. 20, 2022 (DE) 102022134159.3

(51) Int. Cl. A61B 34/37; A61B 90/00

(52) U.S. Cl. CPC A61B 34/37; A61B 90/08

(57) ABSTRACT

The invention relates to a system for controlling at least one functionality of a surgical instrument, comprising: a mode setting unit which is designed to set one of at least two operating modes for the functionality of the surgical instrument and to output an output signal indicating the set operating mode; and a control unit designed to receive the output signal and to control the functionality of the instrument, wherein the control unit is designed to allow the functionality without restriction in the first operating mode and to restrict the functionality in the second operating mode.

### TECHNICAL FIELD OF THE INVENTION

The present invention relates to a system for controlling at least one functionality of a surgical instrument, in particular an articulated and/or steerable instrument, including a motor-driven or robotized instrument. Furthermore, the present invention relates to a computer-implemented method for controlling at least one functionality of a surgical instrument.

### BACKGROUND OF THE INVENTION

Medical instruments, and in particular surgical instruments, have a limited operating service life that defines how long the medical instrument will perform or be useful. At the end of an instrument's service life, the instrument may be repaired or serviced to restore functionality, or the instrument may be replaced with another new instrument.

There are numerous factors that influence the service life or degree of wear of a medical instrument. These include the time of use of the instrument, the degree of tension and stress of each use, and environmental factors, such as the pH value of a body fluid of a patient being treated with the medical instrument.

Especially with articulated instruments, the articulation of the various joints is an important factor that influences the service life of the medical instrument. However, it is difficult for an operator of a surgical instrument to constantly use the medical instrument with an appropriate or suitable loading force. Sometimes it happens that the joints of the medical instrument are subjected to a mechanical force that is higher than the force actually required for the respective surgical procedure. This can result in surgical instruments being exposed to unnecessarily high mechanical stresses. This cumulative effect contributes to an increase in the degree of wear of the surgical instrument and consequently leads to a reduction in the service life of the surgical instrument in question.

Conventional surgical instruments are not equipped with mechanisms that take into account how many functionalities of a surgical instrument are used during a surgical procedure and to what extent or with what intensity these functionalities are used. In particular, the user of a surgical instrument cannot configure this instrument so that the functionalities of planned surgical activities are automatically adapted.

Ideally, a medical instrument should be used optimally in terms of its performance and taking into account its service life in order to conserve resources in hospitals or clinics. It is therefore necessary to optimize the operating life and performance of an instrument based on the planned surgical activities.

### SUMMARY OF THE INVENTION

It is therefore the object of the present invention to provide a system and a method that provides an optimization of the service life and of the performance of a surgical instrument.

The object of the present invention is achieved by a system having the features of claim 1 and by a computer-implemented method having the features of claim 13. Preferred embodiments of the invention having advantageous features are specified in the dependent claims.

According to a first aspect, the invention comprises a system for controlling at least one functionality of a surgical instrument, comprising: a mode setting unit, which is designed to set one of at least two operating modes for the functionality of the articulated surgical instrument and to output an output signal which signals the set operating mode; and comprising a control unit which is designed to receive the output signal from the mode setting unit and control the functionality of the articulated surgical instrument according to the signaled operating mode, wherein the control unit is designed to enable the functionality of the articulated surgical instrument without restriction in a first operating mode and to restrict the functionality of the articulated surgical instrument in a second operating mode.

The term “surgical instruments” comprises medical instruments suitable for surgical interventions and surgical activities. Particularly relevant to the invention are steerable, articulated medical instruments that are operated manually (possibly also motor-driven), such as scissors or endoscopes, or can be controlled by automation systems, such as robots, to assist in surgical procedures.

The functionality of a medical instrument is understood to mean, in particular, a property of the instrument that makes a particular use of the instrument possible. A functionality of scissors as a simple instrument can include, for example, “cutting”. This functionality of the scissors can be exercised with or without restrictions. The scissors have two blades attached to a joint. The blades of the scissors have a certain cutting ability or sharpness. The force applied for example by a robot arm of a robot-to the opposing cutting edges of the scissors to cut a thread situated between them can be variably adjusted. For example, when a thin thread is to be cut, the force exerted when cutting the thread can be deliberately reduced to limit functionality in order to protect the scissors and thus extend the remaining service life of the scissors. The force exerted by the robot arm represents a parameter of the “cutting” functionality of the scissors instrument. Another possible parameter of the “cutting” functionality is, for example, the final angle between the cutting edges of the scissors, especially if the object to be cut or the patient's body tissue is not to be completely severed.

Other examples of possible functionalities of a surgical instrument include “grasping”, “pulling”, “pushing”, “sewing”, “illuminating”, “rinsing” and “irradiating” objects or body tissue of a patient.

A surgical instrument may include a robotic arm. A functionality of the instrument may, for example, be a functionality of a joint of the robot, e.g., a rotation ability or a speed of the robot arm of the instrument. Surgical instruments usually have more than one functionality.

An operating mode for a functionality of an instrument refers to a configuration of the instrument that defines the permitted values of the functionality of the instrument. An operating mode can also represent a configuration that defines permitted values for a plurality of functionalities of the instrument simultaneously.

The mode setting unit and the control unit can each be implemented as a device comprising or consisting of at least one central processing unit (CPU) and/or at least one graphics processing unit (GPU) and/or at least one field programmable gate array (FPGA) and/or at least one application specific integrated circuit (ASIC) and/or any combination of the aforementioned elements. Furthermore, the two units include a programming interface (API) via which signals and information can be exchanged with one another by wired and/or wireless means.

Each element of the system of the invention can further comprise a working memory operatively connected to the at least one CPU and/or a non-volatile memory operatively connected to the at least one CPU and/or the working memory. Each element can be partially and/or fully implemented in a local device and/or partially and/or fully implemented in a remote system, such as a cloud computing platform.

The mode setting unit and the control unit can run software, an app, or an algorithm with different data processing capabilities. Both units can be implemented in hardware and/or software, wired and/or wireless, and any combination thereof. They can further comprise an interface with an intranet or the Internet, with a cloud computing service, with a remote server and/or the like.

According to a second aspect, the invention provides a computer-implemented method for controlling at least one articulated surgical instrument, comprising the steps of:
(a) setting one of at least two operating modes for the surgical instrument; and
(b) controlling the articulated surgical instrument, comprising:
(b1) unrestricted enabling of a functionality of the surgical instrument when the first operating mode is set; and (b2) limiting the functionality of the surgical instrument when the second operating mode is set.

In particular, the computer-implemented method according to the second aspect of the invention can be performed with the system according to the first aspect of the invention. The features and advantages described herein in connection with the system are therefore also applicable to the method and vice versa.

A computer-implemented method is understood to mean any method that has been implemented using a computer, a processor or another computing device, for example by means of a CPU, a GPU, an FPGA, an ASIC, a microprocessor, a desktop PC, a mainframe computer, a server farm and/or the like.

According to a third aspect, the invention provides a computer program product that comprises an executable program code that, when executed, is designed to perform the method according to the second aspect of the present invention.

According to a fourth aspect, the invention provides a non-volatile computer-readable data storage medium that comprises executable program code that is designed to perform the method according to the second aspect of the present invention when executed.

The non-volatile, computer-readable data storage medium can comprise or consist of any type of computer memory, in particular a semiconductor memory, such as, e.g., a solid-state memory. The data storage medium can also comprise or consist of a CD, a DVD, a Blu-ray disc, a USB memory stick or the like.

According to a fifth aspect, the invention provides a data stream that comprises or is designed to generate executable program code that, when executed, is designed so that it performs the method according to the second aspect of the present invention.

An idea underlying the invention is to create a control system with which a surgical instrument, in particular an articulated instrument, can be used in different operating modes. The system is designed to offer a user various operating modes for operating the surgical instrument, so that the operating life of the surgical instrument can be increased by appropriate selection of the operating modes. A mode setting unit is designed to provide a user with a choice of different operating modes or configurations for the surgical instrument, including a first operating mode (or standard operating mode) and a second operating mode (or extended service life operating mode). The selected operating mode is output by an output signal to a control unit, which enables the functionality of the surgical instrument in the standard operating mode without restriction and at least partially limits the functionality of the surgical instrument in the extended service life operating mode in order to protect the surgical instrument.

The control system for articulated surgical instruments described above advantageously allows the implementation of a computer-implemented method to control the functionalities of an articulated surgical instrument. First, an operating mode is set by selecting from at least two operating modes (e.g., a standard mode and an extended service life mode). The articulated instrument is then controlled based on the selected operating mode. In the extended service life mode, the affected functionalities of the instrument are restricted, and in the standard mode, the affected functionalities of the surgical instrument are enabled without restriction.

An advantage of the present invention is that by selecting different operating modes the operating life of a surgical instrument can be fully utilized by a user. Based on the requirements of the respective surgical procedures, the various functionalities of the surgical instrument can be optimally adapted to each situation with regard to the required performance and taking into account the maximum service life of the surgical instrument. This helps the surgical instrument to be used more efficiently.

Another advantage of the invention is that the number of operating modes is not limited. The system of the invention can in principle be equipped with any number of operating modes. It is also conceivable that the control system offers a plurality of instrument-specific operating modes.

Advantageous embodiments and further developments arise from the dependent claims and from the description of the various preferred embodiments shown in the attached figures.

According to some embodiments of the invention, it is provided that the functionality of the surgical instrument be controllable by the control unit with respect to at least one of the following parameters:

Speed of movement and acceleration of the instrument; angle of articulation of the instrument; mechanical forces and/or torques exerted by the surgical instrument; bending limit of the surgical instrument;) current used by the instrument; pressures exerted on the instrument by fluids; and energy discharged by the instrument.

The control unit can set one or more parameters. The setting can be done mechanically or by software. For example, the speed and acceleration of an endoscope can be set to be below a threshold using software. Similarly, the forces exerted by the endoscope can be limited.

The various surgical instruments are usually equipped with sensors (e.g., pressure sensors) that are suitable for determining the parameters and can output the information. The control unit can operate the functionalities of the surgical instrument through various actuators that exert mechanical forces to adapt the values of the parameters to the requirements of the selected operating mode.

According to some embodiments of the invention, it is provided that a third operating mode can also be set by the mode setting unit of the system, wherein the control unit of the system is designed to set at least one parameter of the functionality of the surgical instrument to a specific value in the third operating mode.

Substantially, the system has at least a standard mode, in which the actuators driven by the control unit remain inactive, and an extended service life mode, in which the functionalities of the surgical instrument are optimized in order to extend the service life of the instrument or to minimize the degree of wear of the surgical instrument during its use. The provision of a third operating mode may be advantageous in order to be able to perform particularly challenging surgical tasks with the aid of the surgical instrument. In some applications, an endoscope may need to be rotated repeatedly strongly and quickly. In these applications it is advantageous to provide a high-performance mode. When setting a high-performance mode, the system control unit sets the affected functionalities of the surgical instrument to high values to ensure optimal performance.

According to some embodiments of the invention, it is provided that a fourth operating mode can also be set by the mode setting unit, wherein the control unit is designed to set at least one parameter of the functionality of the surgical instrument to certain task-specific values in the fourth operating mode.

In the fourth operating mode, the functionalities are optimized for the specific method. The optimization can be based on existing data sets from past methods. The data sets may include patient-specific information as well as various procedural data (e.g., data from sensors, endoscopic images, information about the surgical instrument, etc.).

According to some embodiments of the invention, it is provided that the mode setting unit comprise an evaluation unit, which is designed to determine the parameters of the functionality of the surgical instrument using at least one deterministic algorithm.

The adjustment of the various parameters of one or more functionalities of the surgical instrument is facilitated by the application of an algorithm. The algorithm can calculate optimal parameter settings in the extended service life mode, the high-performance operating mode, and the method-specific operating mode of the surgical instrument. For this purpose, an objective function is defined which depends on a weighted combination of the various parameters to be adjusted of the surgical instrument. In the extended service life mode, the objective function can be defined by a mathematical model of the service life of a surgical instrument. Similarly, target functions can be defined for all operating modes. Using such an algorithm, the user of the surgical instrument only needs to select one operating mode to operate the surgical instrument from a group of provided operating modes. The parameters of the functionalities of the surgical instrument determined by the algorithm are adjusted by the control unit and the actuators according to the set operating mode.

According to some embodiments of the invention, the restriction of the functionality of the surgical instrument in the second operating mode is designed to maximize the service life of the surgical instrument. This can be achieved by limiting the values of a functionality of the surgical instrument below a predetermined threshold. Alternatively, an objective function, which is modeled using a model the service life of the surgical instrument, can be maximized by an algorithm.

According to some embodiments of the invention, it is provided that the mode setting unit be further designed to receive a signal about a surgical activity to be performed with the surgical instrument and, based thereon, to provide only predetermined operating modes for the functionality of the surgical instrument.

In highly developed versions of the instrument according to the invention, which provide a plurality of operating modes, it is in many cases advantageous for the mode setting unit to make a preselection of suitable operating modes according to. It may happen that for a specific surgical method performed using the surgical instrument, only the standard operating mode and the high-performance mode of the surgical instrument are offered for selection. This may be useful, for example, for a particularly demanding surgical method for which the use of the surgical instrument in extended service life mode is not suitable. During the method, the user can switch from the standard operating mode to the high-performance mode of the surgical instrument and vice versa, depending on the situation. It is also conceivable that the system according to the invention offers different operating modes of the surgical instrument for the different phases of a surgical activity. For example, an endoscope as an instrument can be operated in the extended service life mode or in the standard mode at the beginning and at the end of an endoscopic procedure, whereas the high-performance mode of the surgical instrument is automatically set, e.g., as soon as a force-intensive activity is required during the endoscopic procedure.

According to some embodiments of the invention, the mode setting unit and/or the control unit are integrated into the surgical instrument. For example, the mode setting unit may have an interface on the instrument, e.g., a user may select the operating mode of the surgical instrument using buttons or a touchscreen. The control unit may also be located in the surgical instrument and is connected, for example, to the instrument's actuators via cables.

According to some embodiments of the invention, it is provided that the mode setting unit be incorporated into a computing device of an automation unit. A centralized mode setting unit may also be provided according to the invention. This can be particularly advantageous in surgical environments where a robot collects data from different data sources (e.g., from instruments, from the patient, from the operating room).

According to some embodiments of the invention, the mode-setting unit is implemented by a server and/or a cloud computing platform. This can be advantageous if different instruments are to be programmed with operating modes at once or simultaneously in order to perform different tasks one after the other. This can be particularly advantageous for instruments that are controlled by a robot. This means that the planned surgical procedures can be carried out remotely by the various robots. Even for patients treated in different hospitals, it is advantageous for the mode setting unit to be implemented in a cloud computing platform. Thus, the mode setting unit can be used to treat patients in different hospitals.

According to some embodiments of the invention, the system is designed for controlling a plurality of functionalities of a surgical instrument. In complex surgical procedures, it is often necessary to consider various functionalities. The system is not limited to handling a specific functionality, but can advantageously handle combinations of functionalities. This allows the system to offer operating modes that are more efficient and flexible.

According to some embodiments of the invention, the system is designed for controlling at least one functionality of a plurality of surgical instruments. The system contains instrument-specific information and can be used for different instruments.

According to some embodiments of the invention, it is provided that the system further comprise an availability determination device which is designed to provide at least one item of information indicating an availability of the functionality of the surgical instrument. The availability of a functionality of a surgical instrument preferably refers to the service life or the degree of wear of the instrument. For this purpose, it is provided that the availability determination device be connected to an availability device, so that they can exchange information with one another. The availability device is designed to calculate the expected service life of the instrument. This information can be retrieved from the availability determination device. After each surgical procedure, the availability determination device can output information to the availability device to update the expected service life of the instrument.

Information about the remaining service life (or “current remaining service life”) of a functionality and/or the instrument may be stored in various locations, wherein multiple memory storage locations are also possible: firstly, in the instrument itself, secondly, centrally in a combined device, and/or thirdly, in a server, such as a cloud storage facility. The service lives of the functionalities of the instrument may be stored in memory and be outputable/readable separately, or a service life of the instrument as a whole may be stored in memory and be outputable/readable, for example formed from the service life of the functionality of the instrument having the shortest service life.

Storing information about the remaining service life in memory (e.g., via an RFID circuit in the instrument) has the advantage that, when the instrument is used on different robots, for example, the service life of the individual instrument currently in use is always known.

Storing centrally in memory in a combined device may, for example, mean storing in the CPU of a robot that guides the instrument (i.e., the combined device is an instrument-robot system). The CPU may be designed to acquire data from the instrument-robot system and the environment, for example from the robot itself, from an (in particular endoscopic) imaging device, from an integrated operations management system, from an integrated hospital information system and/or other similar sources.

Storing data on a server, such as a cloud storage facility, has the advantage that, for example, when using the same instruments with different robots, the service life (of the functionalities) of the instrument can always be precisely tracked.

According to some embodiments, the information indicating availability of the functionality of the surgical instrument is service life information. The service life information may indicate a remaining service life of the functionality and/or the instrument, and may be stored in a data memory of the system (e.g., a data memory of the availability determination device) and be readable from there.

Particularly if the instrument is part of the system, the service life information can preferably be stored in a data memory of the instrument and be readable from there. In this way, the service life information is always present on the instrument itself and can be transported or moved with the instrument to different locations or sites of use without the service life information having to be reassigned. The service life information can be stored in memory on the instrument, for example in an RFID circuit, so that the service life information can be conveniently read out on the instrument via RFID communication.

Although some functions are described here and below as being performed by devices, this does not necessarily mean that these devices are provided as separate entities. In cases where one or more devices or even a part thereof are provided as software, the devices can be implemented by sections or snippets of program code, which can be separate from one another, but can also be interwoven or integrated with one another.

Similarly, in cases where one or more devices are provided as hardware, the functions of one or more devices can be provided by one and the same hardware component, or the functions of a plurality of devices can be distributed among a plurality of hardware components that do not necessarily correspond to the devices. It is therefore to be assumed that any application, system, process, etc. that has all the features and functions attributed to a particular device comprises or implements that device. In particular, it is possible that all devices are implemented by program code executed by, e.g., a server or a cloud computing platform.

All mentioned embodiments and implementations can be combined with one another as desired, to the extent that this is sensible.

The further scope of applicability of the present method and system becomes apparent from the following figures, the detailed description and the claims. It should be understood, however, that the detailed description and specific examples, while indicating preferred embodiments of the invention, are primarily intended for purposes of illustration and that various changes and modifications within the basic ideas and scope of the invention will become apparent to a person skilled in the art.

### BRIEF DESCRIPTION OF THE DRAWINGS

The invention will now be described with reference to its advantageous embodiments based on the following drawings. In the drawings, identical or functionally similar elements are designated by the same reference numerals in the individual views. The drawings serve to further illustrate embodiments of concepts incorporating the claimed invention and to explain various principles and advantages of those embodiments. Elements depicted in the drawings are not necessarily shown to scale. This serves to clearly and distinctly disclose the bases and principles of the invention.

### IN THE DRAWINGS

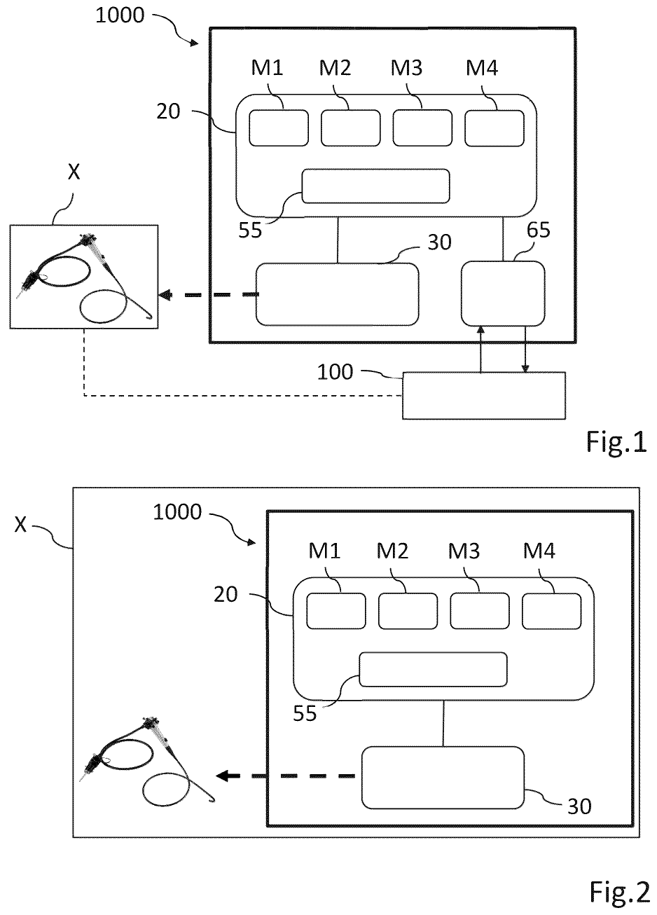

FIG. 1 shows a system for controlling at least one functionality of a surgical instrument according to an embodiment of the invention;

FIG. 2 shows a system for controlling at least one functionality of a surgical instrument according to a further embodiment of the invention;

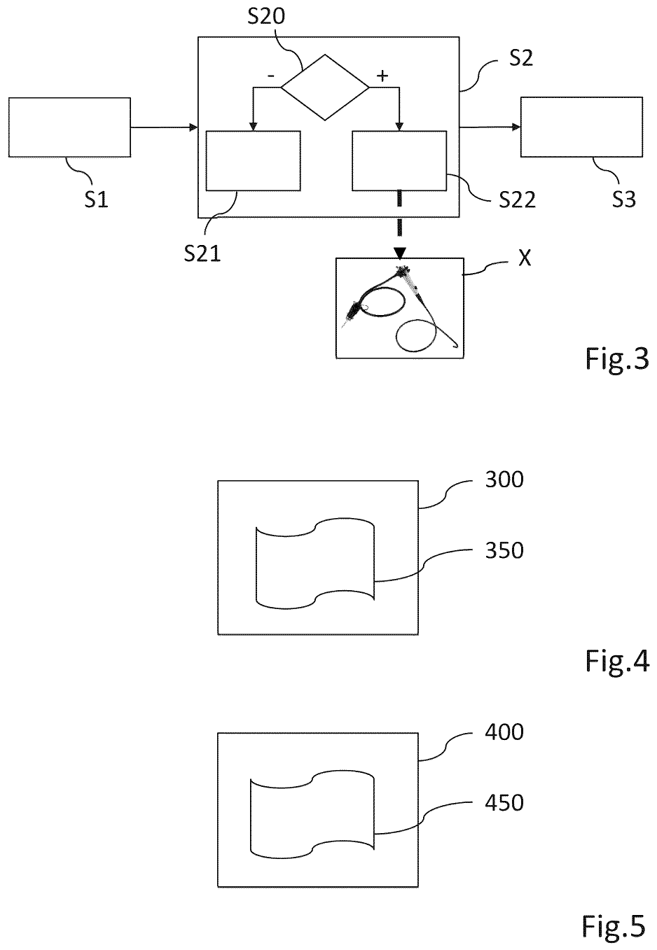

FIG. 3 shows in a schematic block diagram a representation of the sequence of a computer-implemented method for controlling at least one functionality of a surgical instrument according to an embodiment of the invention;

FIG. 4 is a schematic block diagram that illustrates a computer program product according to an embodiment of the third aspect of the present invention; and

FIG. 5 is a schematic block diagram that shows a non-volatile computer-readable data storage medium according to an embodiment of the fourth aspect of the present invention

In some cases, well-known structures and devices are shown in block diagram form in order to illustrate the concepts of the present invention. The numbering of the steps in the method is also intended to facilitate their description. They do not necessarily imply a particular order of steps. In particular, several steps can be carried out simultaneously.

### DESCRIPTION OF THE DRAWINGS

The detailed description of the accompanying drawings contains specific details in order to provide a thorough understanding of the present invention. However, it will be apparent to a person skilled in the art that the present invention may also be executed without these specific details.

FIG. 1 shows a system 1000 for controlling at least one functionality of a surgical instrument X according to a possible embodiment of the invention. The various components and functions are schematically represented as blocks. The spatial arrangement of the blocks in FIG. 1 serves only to illustrate the embodiment shown. The instrument X is shown in FIG. 1 as an endoscope that can possibly be controlled using a robot. For illustration purposes, such an endoscope is used below as a concrete example of an instrument X. However, the principles of the invention apply to any surgical instruments, such as, e.g., scissors or scalpels, and also to any instruments whose handling can be assisted by robotics, as well as to robots as instruments themselves.

As shown in FIG. 1, the system 1000 comprises a mode setting unit 20, a control unit 30 and an availability determination device 65.

The mode setting unit 20 is designed to set at least two operating modes for one or more functionalities of the endoscope X. The functionalities of the endoscope X can include, for example, the freedom of movement (in particular permissible angles of movement of the joints) or the articulation of the various joints of the endoscope X. For example, four operating modes are shown in FIG. 1: a standard mode M1, an extended service life mode M2, a high-performance mode M3 and a method-specific mode M4. The mode setting unit 20 is further designed to output an output signal indicating the selected operating mode. The operating mode of the surgical instrument can be selected by a user based on the surgical activity to be performed and taking into account optimal use of the endoscope X in terms of its performance and service life. This allows the instrument's operating life to be optimized.

The control unit 30 is designed to receive the output signal of the mode setting unit 20 and to control the functionality of the instrument X. This can be achieved using various actuators. If the selected operating mode is the standard mode M1, the actuators remain inactive. The endoscope X then operates under normal conditions. If the selected operating mode is the extended service life mode M2, various parameters of the surgical instrument are automatically adjusted, including the speed of movement and acceleration of the endoscope X; the angles of the various articulations of the endoscope X; the applied forces and torques exerted by the endoscope X; an electrical current consumed by the endoscope X when performing the surgical activity; the external pressures exerted on the endoscope X, e.g., by the patient's bodily fluids; or the energy released by the endoscope X.

The parameters can be set using software. For example, using software, the articulation of endoscope X can be determined to be below a threshold. Similarly, the forces exerted by the endoscope X can be limited. The control unit can operate the functionalities of the surgical instrument through various actuators that exert mechanical forces to adapt the values of the functionalities to the requirements of the selected extended service life mode M2.

In the high-performance mode M3, some of the aforementioned parameters are adjusted to maximize the performance of the endoscope X, while in the method-specific mode M4, the setting of the parameters of the functionalities of the surgical instrument is automatically adapted to a specific surgical procedure to be performed. It is therefore conceivable that the method-specific mode M4 comprises a combination of other operating modes in a specific order based on the characteristics of the surgical activity.

For illustration, the endoscope X in FIG. 1 can be used, for example, to perform endoscopic foreign body removal. The foreign body, e.g., a button, is located in the stomach of a patient. The surgical procedure substantially consists of inserting the endoscope X into the patient's esophagus up to the stomach, removing the button, and removing the endoscope X. In this case, a user can set the extended service life mode M2 during the insertion of the endoscope X. This limits the speed of the endoscope X. When removing the button, the standard mode MI or even the high-performance mode M3 can be selected, by which, for example, the acceleration of the endoscope X and the force exerted by a forceps of the endoscope X are set to a high value in order to retrieve and extract the button from the stomach. When removing the endoscope X, the user can select, for example, the standard mode MI or the extended service life mode M2.

Alternatively, the system 1000 according to the invention can be used to select a method-specific mode M4 for the endoscope X, which is particularly suitable for foreign body removal. This mode has a sequence of parameter settings that optimize the service life and performance of the endoscope X during foreign body removal.

The mode setting unit 20 of FIG. 1 comprises an evaluation unit 55 which is designed to determine the parameters of the required functionalities of the endoscope X using a deterministic algorithm. The algorithm depends on which operating mode M1-M4 is selected. In the extended service life mode M2, the target function that the algorithm maximizes is the operating life of the endoscope X. In the high-performance mode M3, however, the target function that the algorithm maximizes is the performance of the endoscope X. The different target functions consist of weighted combinations of the parameters.

FIG. 1 further shows an availability determination device 65, which is designed to provide at least one piece of information which determines the expected remaining service life and/or the current degree of wear of the endoscope X. The availability determination device 65 comprises at least a central processing unit (CPU), a data memory and an application programming interface (API). The availability determination device 65 is connected to an external availability device 100 such that they can mutually exchange information. The availability device 100 is designed to calculate the remaining service life of the endoscope X. This calculation can be based on the number of uses of endoscope X. Alternatively, the remaining service life can be calculated using sophisticated methods based on sensor data.

The service life of the endoscope X calculated by the availability device 100 is retrieved by the availability determination device 65. After removal of the foreign body, the availability determination device 65 may output information to the availability device 100, so that the availability device 100 can update the remaining service life of the endoscope X. For example, if the availability device 100 is a conventional device that counts cycles (the number of uses) of the endoscope X, then the availability determination device 65 may cause the number to increase by one unit (in case of normal use), increase by two units (in case of intensive use), or not increase the number (in case of below-average intensive use). The updated remaining service life may, for example (if necessary additionally), be stored in a data memory of the availability determination device 65, in particular in the endoscope X itself, for example in such a way that it can be retrieved or read out using RFID technology.

FIG. 2 shows a system 1000 for controlling at least one functionality of a surgical instrument X in a further embodiment of the invention.

In contrast to FIG. 1, in which the system 1000 and the endoscope X are shown as separate, in FIG. 2 the system 1000 is implemented and integrated in the endoscope X. The operating mode can then be selected by the user via a user interface, e.g., using a keyboard or a touchscreen.

In FIG. 2, the entire system 1000 is integrated into the endoscope X for the sake of simplicity. However, it is conceivable that only some of its elements are located in the endoscope X, while other elements are located elsewhere, i.e., separate from the endoscope X. Thus, the system 1000 may comprise the endoscope X or vice versa. In some embodiments, the evaluation unit 55 may be provided outside the endoscope X, for example in a cloud computing platform. Similarly, the availability determination device 65 may be implemented either in a server or in a cloud computing platform.

FIG. 3 shows in a schematic block diagram a representation of the sequence of a computer-implemented method for controlling at least one functionality of a surgical instrument X according to an embodiment of the invention. The method may advantageously be carried out with the system 1000 shown in FIG. 1.

In a step S1, an operating mode of at least two available operating modes is set for the instrument X. The operating mode may, for example, be selected by a user with regard to the performance required by the instrument, taking into account an optimization of the (operating) life of the instrument X, to perform certain surgical activity.

In a step S2, the instrument X is controlled in the selected operating mode by limiting one or more functionalities of the instrument X, either by software or by a mechanical action. In the embodiment of the invention shown in FIG. 3, the step S2 comprises the steps S20, S21 and S22. In step S20, it is determined whether the selected operating mode is the first operating mode (standard mode) M1 or not. If the set operating mode is the standard mode M1 (marked with a−), then in step S21 the functionality of the instrument is enabled without restrictions. If the set operating mode is another operating mode (marked with a+), for example the extended service life mode M2, the high-performance mode M3 or the method-specific mode M4 shown in FIGS. 1 and 2, then in a step S22 the functionality of the instrument X is restricted accordingly.

In a further step S3, the calculation of the instrument's service life is updated according to the surgical activity performed. Information about the surgical activity performed by the surgical instrument is output to an availability device, which is designed to calculate the remaining service life or degree of wear of the instrument X. Depending on how intensively the surgical activity is performed, the remaining service life of the surgical instrument is calculated and a corresponding service life value is updated.

FIG. 4 shows a schematic block diagram that illustrates a computer program product 300 according to an embodiment of the third aspect of the present invention. The computer program product 300 comprises an executable program code 350 that, when executed, is configured to perform the method according to any embodiment of the second aspect of the present invention, in particular as described in the preceding figures.

FIG. 5 shows a schematic block diagram that illustrates a non-volatile computer-readable data storage medium 400 according to an embodiment of the fourth aspect of the present invention. The data storage medium 400 comprises an executable program code 450 that, when executed, is configured to perform the method according to any embodiment of the second aspect of the present invention, in particular as described with reference to the preceding figures.

The non-volatile, computer-readable data storage medium can comprise or consist of any type of computer memory, in particular a semiconductor memory, such as a solid-state memory. The data storage medium can also comprises or consist of a CD, a DVD, a Blu-ray disc, a USB memory stick or the like.

1-17. (canceled)

18. A system for controlling at least one functionality of a surgical instrument, comprising:
a mode setting unit to set one of at least two operating modes for the functionality of the surgical instrument and to output an output signal to set the operating mode of the surgical instrument; and
a control unit to receive the output signal and to control the functionality of the surgical instrument in accordance with the signaled operating mode, wherein the control unit is designed to allow the functionality of the surgical instrument without restriction in the first operating mode and to restrict the functionality of the surgical instrument in the second operating mode.

19. The system of claim 18, wherein the functionality of the surgical instrument is controllable by the control unit with respect to at least one of the following parameters:
speed of movement and/or acceleration of the surgical instrument;
angle of articulation of a joint of the surgical instrument;
forces and/or torques exerted by the surgical instrument or on the surgical instrument;
bending limit of the surgical instrument;
an energy consumed by the surgical instrument when performing the surgical activity, in particular an electric current consumed;
pressures exerted on the surgical instrument by liquids; and
energy released by the surgical instrument when performing the surgical activity.

20. The system of claim 19, wherein a third operating mode is set by the mode setting unit, wherein the control unit sets at least one parameter of the functionality of the surgical instrument to a specific value in the third operating mode.

21. The system of claim 20, wherein a fourth operating mode is set by the mode setting unit, wherein the control unit sets at least one parameter of the functionality of the surgical instrument to certain task-specific values of the surgical activity to be performed in the fourth operating mode.

22. The system of claim 19, wherein the mode setting unit comprises an evaluation unit to determine the at least one of the parameters of the functionality of the surgical instrument using at least one deterministic algorithm.

23. The system of claim 18, wherein the restriction of functionality in the second operating mode is to maximize the expected remaining operating life of the surgical instrument.

24. The system of claim 18, wherein the mode setting unit receives a signal indicating a surgical activity to be performed with the surgical instrument and, based thereon, provides only corresponding associated operating modes for the functionality of the surgical instrument.

25. The system of claim 18, wherein at least one of the mode setting unit or the control unit are integrated into the surgical instrument.

26. The system of claim 18, further comprising:
an automation unit comprising a computing device; and
wherein the computing device comprises the mode setting unit.

27. The system of claim 18, wherein the mode-setting unit is implemented by at least one of a server or a cloud computing platform.

28. The system of claim 18, wherein the system for controlling at least one functionality of the surgical instrument controls a plurality of functionalities of the surgical instrument.

29. The system of claim 18, wherein the system for controlling at least one functionality of a plurality of surgical instruments that comprise at least the surgical instrument.

30. The system of claim 1, wherein the system further comprises an availability determination device which is to provide at least one item of information indicating an availability of the functionality of the surgical instrument.

31. The system of claim 30, wherein at least one item of information indicating the availability of the functionality of the surgical instrument comprises service life information indicating a remaining service life of at least one of the functionality of the surgical instrument or the surgical instrument, and wherein the service life information is stored in a data memory of the system.

32. The system of claim 31, wherein the service life information is stored in the data memory of the system comprising a data memory of the surgical instrument.

33. A computer-implemented method for controlling at least one surgical instrument, comprising the steps of:
setting one of at least two available operating modes for the surgical instrument; and
controlling the surgical instrument, comprising:
unrestricted enabling of a functionality of the surgical instrument when the first operating mode is set; and
limiting the functionality of the surgical instrument when the second operating mode is set.

34. The computer-implemented method of claim 33, wherein the functionality of the surgical instrument comprises at least one of:
speed of movement and/or acceleration of the surgical instrument;
angle of articulation of a joint of the surgical instrument;
forces and/or torques exerted by the surgical instrument or on the surgical instrument;
bending limit of the surgical instrument;
an energy consumed by the surgical instrument when performing the surgical activity, in particular an electric current consumed;
pressures exerted on the surgical instrument by liquids; and
energy released by the surgical instrument when performing the surgical activity.

35. The computer-implemented method of claim 34, further comprising setting at least one parameter of the functionality of the surgical instrument, by a third operating mode, to a specific value in the third operating mode.

36. The computer-implemented method of claim 35, further comprising setting at least one parameter of the functionality of the surgical instrument, by a fourth operating mode, to a specific value in the fourth operating mode.

37. A surgical instrument, comprising:
a mode setting unit to set one of at least two operating modes for the functionality of the surgical instrument and to output an output signal to set the operating mode of the surgical instrument; and
a control unit to receive the output signal and to control the functionality of the surgical instrument in accordance with the signaled operating mode, wherein the control unit is designed to allow the functionality of the surgical instrument without restriction in the first operating mode and to restrict the functionality of the surgical instrument in the second operating mode.
